(19) United States
(12) Patent Application Publication
(10) Pub. No.: US 2007/0093519 A1
(43) Pub. Date: Apr. 26, 2007

(54) ANTI-EMETIC USES OF CANNABINOID ANALOGS

(75) Inventor: Bobby W. Sandage JR., Lexington, MA (US)

Correspondence Address: FOLEY AND LARDNER LLP;SUITE 500, 3000 K STREET NW, WASHINGTON, DC, 20007, US

(73) Assignee: Indevus Pharmaceuticals, Inc.

(21) Appl. No.: 11/534,156

(22) Filed: Sep. 21, 2006

Related U.S. Application Data
(60) Provisional application No. 60/719,204, filed on Oct. 20, 2005.

(51) Int. Cl. A61K 31/473; A61K 31/353

(52) U.S. Cl. 514/290; 514/454

(57) ABSTRACT

The present invention relates to non-psychoactive cannabinol analogs of tetrahydrocannabinol, (3R,4R)-Δ⁸—THC-11-oic acids, for treating and preventing nausea and relieving symptoms thereof.

### CROSS-REFERENCE TO RELATED PATENT APPLICATION

This application claims priority to United States provisional application no. 60/719,204 filed Oct. 20, 2005 the disclosure of which has been incorporated by reference herein in its entirety.

### FIELD OF THE INVENTION

The present invention relates to the treatment of nausea using non-psychoactive derivatives of tetrahydrocannabinol.

### BACKGROUND OF THE INVENTION

1. THC Derivatives

1. THC Derivatives

Δ⁹-Tetrahydrocannabinol (THC]), is the major psychoactive constituent of marijuana.

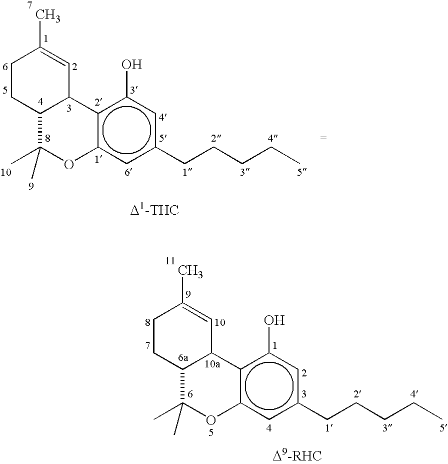

In addition to mood-altering effects, THC exhibits other activities which may have therapeutic value. The potential therapeutic value of THC has led to a search for related compounds which, while devoid of psychoactive effects, retain the activities of potential medicinal value.

Previous work with Δ⁸-tetrahydrocannabinol [(3R,4R) 6a,7,10,10a-tetrahydro-6,6,9-trimethyl-3-pentyl-6H-dibenzo[b,d]pyran-1-ol, hereinafter referred to as Δ⁸—THC], which is depicted below in Formula II, has indicated that certain derivatives of this compound may prove clinically useful.

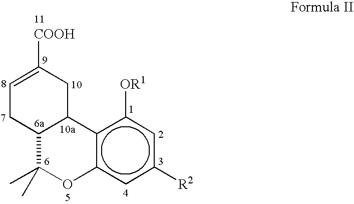

The 11-carboxy derivative of Δ⁸—THC [Δ⁸—THC-11-oic acid] has been reported to be a non-psychoactive, potent antagonist to endogenous platelet activating factor and, thus, a useful treatment for PAF-induced disorders, such as asthma, systemic anaphylaxis, and septic shock. (See U.S. Pat. No. 4,973,603, incorporated herein by reference.) Another derivative, (3S,4S)-11-hydroxy-Δ⁸—THC-1′,1′ dimethylheptyl, essentially free of the (3R,4R) form, has been reported to possess analgesic and anti-emetic activities. (See U.S. Pat. No. 4,876,276, also incorporated herein by reference.)

2. Nausea

Nausea and vomiting occur for many reasons. The most common causes are motion sickness, which may occur in many settings including travel by car, air, or boat, and can last a few hours to a few days. Viral infections can cause nausea and vomiting, as can food poisoning More than 250 different diseases can cause food poisoning. The most common diseases are infections caused by bacteria, such as campylobacter, salmonella, shigella, E. coli, listeria and botulism.

Some medications can cause nausea, such as anti-cancer drugs and morphine, as well as radiation therapy for cancer.

Many antiemetics have been developed to decrease the severity of nausea. Meclizine hydrochloride (Bonine®) is an antihistamine that is effective in the treatment of nausea, vomiting, and dizziness associated with motion sickness. It should not be taken by people with lung diseases, glaucoma, or difficulty with urination due to an enlarged prostate unless recommended by a physician. Meclizine may cause drowsiness and should not be taken with other medicines having sedative side effects such as alcohol, tranquilizers, or sleeping pills. Due to drowsiness, persons using meclizine should not drive or operate dangerous machinery. Meclizine is not recommended in children under 12 or in pregnant or nursing females unless recommended by a doctor.

Dimenhydrinate (Dramamine®) also is an antihistamine. Its use should be limited to motion sickness. Due to the potential for causing drowsiness, dimenhydrinate should be avoided in the same situations as Meclizine.

Emetrol® is an oral solution designed to soothe the stomach when nausea and vomiting are caused by a viral or bacterial infection or overeating. Emetrol contains sugar and phosphoric acid. Diabetics should not use Emetrol without medical supervision because of the concentrated sugar. According to its manufacturer, Emetrol should not be taken for more than five doses in one hour without consulting a physician. A doctor should also be consulted when considering using this medicine for pregnant or nursing women and young children.

None of the currently used treatments for nausea is capable of fully relieving the symptoms in all cases. Patients frequently combine different treatments in an attempt to address all of their symptoms. Clearly, although numerous treatments have been developed in an attempt to control nausea there is still a great need in the art for effective treatments.

3. Description of Related Art

U.S. Pat. No. 5,338,753 discloses (3R,4R)-Δ⁶—THC-7-oic acids (which correspond to (3R,4R)-Δ⁸—THC-11-oic acids, but were named using an alternative numbering system) that are useful as anti-inflammatory agents and analgesics, as well as methods of synthesizing them, but does not disclose compositions or methods for treating patients suffering from nausea.

U.S. Pat. Nos. 6,162,829 and 6,355,650 disclose derivatives of (3R,4R)-Δ⁸—THC-11-oic acids that are also useful as anti-inflammatory agents and analgesics, and methods of synthesizing them. They do not disclose compositions or methods for treating patients suffering from nausea.

U.S. Pat. No. 6,448,288 discloses the use of Δ⁸—THC-11-oic acids to decrease cell proliferation, but fails to disclose compositions or methods for treating patients suffering from nausea.

U.S. Published Application No. 2004/0054007 discloses methods for decreasing cell proliferation using (3R,4R)-Δ⁸—THC-11-oic acid, but it also fails to disclose compositions or methods for treating patients suffering from nausea.

U.S. Published Application No. 2004/0225011 discloses methods of using cannabinoid compounds that are derivatives of THC to decrease cell proliferation, and does not disclose compositions or methods for treating patients suffering from nausea.

The disclosures of each of these patents and published applications are incorporated herein by reference in their entirety.

It is desired, however, to provide a method of treating, alleviating, and/or relieving symptoms associated with nausea by use of the Δ⁸—THC-11-oic acid derivatives, such as those described above, as well as pharmaceutical compositions suitable for such use.

### SUMMARY OF THE INVENTION

One object of the present invention to provide compositions and methods for treating a patient suffering from nausea, whereby the cannibinol analogs and analogs of (3R,4R)-Δ8—THC-11 -oic acids according to the present invention are administered to said patient.

According to a first aspect of the present invention, unique methods are provided for the treatment of nausea in a mammal using a compound having Formula 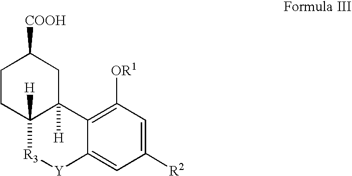
wherein R¹ is hydrogen, —COCH₃ or —COCH₂CH₃; R² is a branched C₅-C₁₂ alkyl group, which may optionally have a terminal aromatic ring, or optionally a branched —OCHCH₃(CH₂)ₘ alkyl group which may have a terminal aromatic ring, wherein m is 0 to 7; R³ is hydrogen, a C₁₋₈ alkyl group, or a C₁₋₈ alkanol group; and Y is nil or a bridging group of NH or oxygen; provided that where Y is oxygen and R² is a branched C₅-C₁₂ alkyl compound, R³ is not —CHCH₃. The method comprises the steps of identifying a mammal suffering from soon to be suffering nausea and administering to the mammal an effective amount of a compound of formula III, or a pharmaceutically acceptable salt, ester, or solvate thereof.

According to a second aspect of the invention, unique compositions and methods are provided for use in treating nausea in a mammal, particularly humans, including a therapeutically effective amount of a compound having Formula III 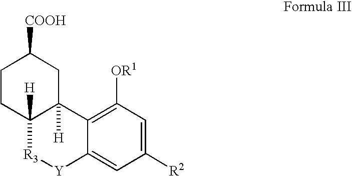
wherein R¹ is hydrogen, —COCH₃ or —COCH₂CH₃; R² is a branched C₅-C₁₂ alkyl group, which may optionally have a terminal aromatic ring, or optionally a branched —OCHCH₃(CH₂)ₘ alkyl group which may have a terminal aromatic ring, wherein m is 0 to 7; R³ is hydrogen, a C₁₋₈ alkyl group, or a C₁₋₈ alkanol group; and Y is nil or a bridging group of NH or oxygen; provided that where Y is oxygen and R² is a branched C₅-C₁₂ alkyl group, R³ is not —CHCH₃; or a pharmaceutically acceptable salt, ester, or solvate thereof The pharmaceutical composition may optionally include a therapeutically effective amount of one or more compounds selected from the group consisting of sodium pentosanpolysulfate, antihistamines, antidepressants, imipramine, antispasmodics, urinary anesthetics, and capsaicin. The pharmaceutical composition may also optionally include a therapeutically effective amount of an anticholinergic agent selected from the group consisting of anisotropine, aprophen, artane, atropine, belladonna, benactyzine, benztropine, clidinium, dicyclomine, glycopyrrolate, homatropine, hyoscyamine, isopropamide, mepenzolate, methantheline, methscopolamine, oxybutynin, oxyphencyclimine, propantheline, scopolamine, terodiline, tridihexethyl, trihexyphenidyl, and trospium.

According to a third aspect of the present invention, unique compositions and methods are provided for a pharmaceutical composition for use in treating nausea in a mammal, including an effective amount of a compound of Formula IV 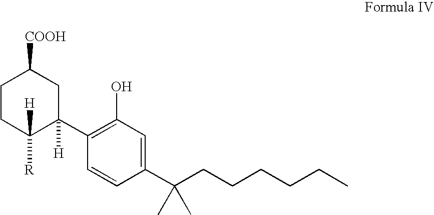
wherein R is hydrogen, a branched or unbranched C₁₋₈ alkyl group, or a branched or unbranched C₁₋₈ alkanol group; a pharmaceutically acceptable salt, ester, or solvate thereof. In one embodiment, R is methyl, ethyl or methanol, or a branched or unbranched, propyl, ethanol, or propanol.

According to a fourth aspect of the present invention, unique compositions and methods are provided for a pharmaceutical composition for use in treating nausea in a mammal including an effective amount of a compound having Formula V 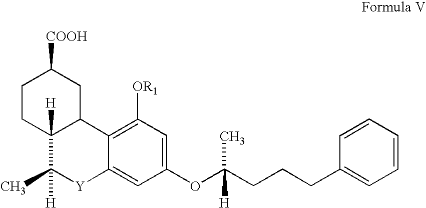
wherein R¹ is hydrogen, —COCH₃ or —COCH₂CH₃, and Y is NH or oxygen, or a pharmaceutically acceptable salt, ester, or solvate thereof.

In another aspect of the invention, subjects suffering from or expected to suffer from nausea are treated by administering an effective amount of at least one compound selected from the compound of Formula III, IV, or V, or a pharmaceutically acceptable salt, ester, or solvate thereof.

### BRIEF DESCRIPTION OF THE DRAWINGS

FIG. 1 illustrates a synthetic scheme for the preparation of analogs of Δ⁸—THC-11-oic acids that are useful in treating nausea in accordance with the present invention.

FIG. 2 illustrates an alternative synthetic scheme for the preparation of analogs of Δ⁸—THC-11-oic acids that are useful in treating nausea in accordance with the present invention.

### DETAILED DESCRIPTION OF THE INVENTION

1. Introduction

The present invention relates to cannabinol analogs, particularly analogs of (3R,4R)-Δ⁸—THC-11-oic acids, as well as to anti-emetic compositions comprising therapeutically effective amounts of these compounds, and methods for treating nausea in a mammal by administering such compounds. The THC derivatives of the present invention have reduced or no psychoactivity and do not bind to the CB1 receptor.

2. Compositions

The present invention relates to compositions and pharmaceuticals useful in relieving symptoms of nausea, which comprise cannabinol analogs, and analogs of Δ⁸—THC-11-oic acids. An illustrative Δ⁸—THC-11-oic acid analog in accordance with the present invention shown below in Formula III, 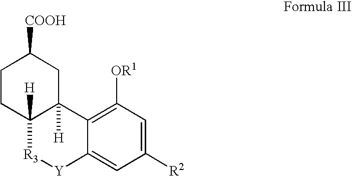
wherein R¹ is hydrogen, —COCH₃ or —COCH₂CH₃; R² is a branched C₅-C₁₂ alkyl group, which may optionally have a terminal aromatic ring, or optionally a branched —OCHCH₃(CH₂)ₘ alkyl group which may have a terminal aromatic ring, wherein m is 0 to 7; and R³ is hydrogen, a C₁₋₈ alkyl or a C₁₋₈ alkanol group; and Y is nil or a bridging group of NH or oxygen; provided that where Y is oxygen and R² is a branched C₅-C₁₂ alkyl, R³ is not —CHCH₃.

In some embodiments, R¹ is hydrogen, R² is 1′,1′-dimethylheptyl, and Y is nil. Thus, in this form, the compounds have Formula IV below:

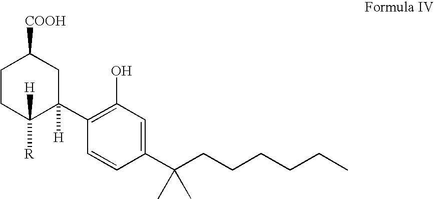

In these compounds, R includes hydrogen, branched or unbranched C₁₋₈ alkyl, and branched or unbranched C₁₋₈ alkanol groups. In some embodiments, R is methyl, ethyl or methanol, or a branched or unbranched, propyl, ethanol, or propanol.

In other embodiments, R² is a branched —OCHCH₃(CH₂)ₘ alkyl compound terminated with a phenyl ring, wherein m is 0 to 7, Y is NH or oxygen, and R³ is —CHCH₃. In yet other embodiments, m is 3, and these compounds have Formula V below:

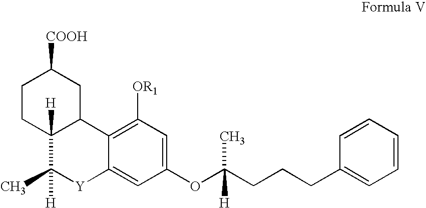

In these compounds, R¹ can be hydrogen, —COCH₃, or COCH₂CH₃, and, for example, R¹ is hydrogen.

The phrase “therapeutically effective amount” means that amount of the pharmaceutical composition that provides a therapeutic benefit in the treatment, prevention, or management of nausea.

Dosage amounts for the cannabinol analogs and analogs of (3R,4R)-Δ⁸—THC-11-oic acids according to the present invention, when administered orally for the relief of symptoms of IC, are generally between about 1 mg and about 200 mg, for example between about 10 mg and about 100 mg per day, or between about 20 mg and about 60 mg per day, administered about 2 to about 4 times daily. As would be understood by one skilled in the art, the dose, and dose frequency, will vary according to the patient's age, body weight, and therapeutic response, as well as the severity of the condition. Typically at a dose of 0.1 mg/kg to 10 mg/kg body weight, typically about 1 mg/kg is given.

The orally administered compounds and pharmaceutical compositions according to the present invention may be optionally administered in conjunction with existing treatments for nausea that are administered orally, via IV or any other route of administration.

The compositions of the present invention may be optionally administered in conjunction with existing treatments for nausea, including, but not limited to, meclizine, Emetrol®, antihistamines such as hydroxizine (Atarax® Vistaril®) and dimenhydrinate.

The orally administered compounds and pharmaceutical compositions according to the present invention may also be optionally administered in conjunction with existing treatments for nausea that are administered via IV.

According to one embodiment of the invention, the compositions may optionally be administered in conjunction with an anticholinergic agent to inhibit the transmission of parasympathetic nerve impulses and thereby reduce spasms of smooth muscle. The anticholinergic agent can be administered orally or via IV, although other routes of administration are contemplated. Such anticholinergic agents may be selected from anisotropine, aprophen, artane, atropine, belladonna, benactyzine, benztropine, clidinium, dicyclomine, glycopyrrolate, homatropine, hyoscyamine, isopropamide, mepenzolate, methantheline, methscopolamine, oxybutynin, oxyphencyclimine, propantheline, scopolamine, terodiline, tridihexethyl, trihexyphenidyl, and trospium.

The pharmaceutical compositions of the present invention may include the active ingredients described above and pharmaceutically acceptable carriers, excipients and the like, and optionally, other therapeutic ingredients. For instance, the drug may be suspended in a vegetable oil, such as olive oil or peanut oil, and, optionally encapsulated in a gelatin capsule. For human therapy, the compounds or pharmaceutical compositions can be administered orally, in the form of a gelatin capsule, or by IV in the form of a suspension or solution.

The term “pharmaceutically acceptable salt” refers to a salt prepared from pharmaceutically acceptable non-toxic acids or bases, including inorganic and organic compounds. Illustrative salts of Formula II include sodium, potassium and ammonium.

Examples of inorganic bases, for potential salt formation include metallic salts made from aluminum, calcium, lithium, magnesium, potassium, sodium, and zinc. Appropriate organic bases may be selected, for example, from N,N-dibenzylethylenediamine, chloroprocaine, choline, diethanolamine, ethylenediamine, meglumaine (N-methylglucamine), and procaine. The compounds and pharmaceutical compositions of the present invention may be administered in the form of such pharmaceutically acceptable salts.

The compounds of interest may also be administered in the form of esters, e.g., methyl, ethyl and the like. Solvates of the compounds of interest may also be useful, including hydrates and the like. Examples of inorganic acids are hydrochloric, hydrobromic, hydroiodic, sulfuric, and phosphoric. Appropriate organic acids may be selected, for example, from aliphatic, aromatic, carboxylic and sulfonic classes of organic acids, examples of which are formic, acetic, propionic, succinic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic, stearic, sulfanilic, algenic, and galacturonic.

The compounds of the present invention may also be included in formulations such as suspensions, solutions and elixirs; aerosols; or carriers such as starches, sugars, microcrystalline cellulose, diluents, granulating agents, lubricants, binders, disintegrating agents, and the like, in the case of oral solid preparations (such as powders, capsules, and tablets), with oral solid preparations being exemplary. An exemplary oral solid preparations are capsules.

Because of their ease of administration, tablets and capsules represent the most advantageous oral dosage unit form, in which case solid pharmaceutical carriers are employed. If desired, tablets may be coated by standard aqueous or nonaqueous techniques. Because of the benefits of IV for relieving symptoms of nausea, IV formulations are another exemplary dosage form, in which case the compounds and pharmaceutical compositions of the present invention are provided dissolved or suspended in a pharmaceutically acceptable solvent or diluent.

In addition to the dosage forms set out above, the compounds and pharmaceuticals of the present invention may also be administered by controlled release means and/or delivery devices such as those described in U.S. Pat. Nos. 3,845,770; 3,916,899; 3,536,809; 3,598,123; and 4,008,719, the disclosures of which are hereby incorporated by reference.

3. Methods

The compounds and pharmaceutical compositions of the present invention can be used in methods of treating mammals suffering from nausea, in both veterinary medicine and human therapy contexts. The method of administering the compounds and pharmaceutical compositions in the acute or chronic management of nausea will vary with the severity of the condition and the route of administration. The dose, and perhaps the dose frequency, will also vary according to the age, body weight, and response of the individual patient. The actual amounts of the active ingredients administered will vary with each case, according to the species of mammal, the nature and severity of affliction being treated, and the method of administration.

The compounds may be administered via any appropriate route, e.g. intravenously, intraarterially, topically, by injection, intraperitoneally, intrapleurally, orally, subcutaneously, intramuscularly, sublingually, intraepidermally, or rectally. Typical methods of administration are orally and via IV. The oral formulations may be solutions, suspensions, suppositories, tablets, granules, powders, capsules, ointments, or creams. The IV formulations may be solutions or suspensions, including compositions comprising liposomes. In the preparation of the pharmaceuticals, a solvent (e.g., water or physiological saline), solubilizing agent (e.g., ethanol, Polysorbates, or Cremophor EL7), agent for making isotonicity, preservative, antioxidizing agent, excipient (e.g., lactose, starch, crystalline cellulose, mannitol, maltose, calcium hydrogen phosphate, light silicic acid anhydride, or calcium carbonate), binder (e.g., starch, polyvinylpyrrolidone, hydroxypropyl cellulose, ethyl cellulose, carboxy methyl cellulose, or gum arabic), lubricant (e.g., magnesium stearate, talc, or hardened oils), or stabilizer (e.g., lactose, mannitol, maltose, polysorbates, macrogols, or polyoxyethylene hardened castor oils) can be added. If necessary, glycerin, dimethylacetamide, 70% sodium lactate, a surfactant, or a basic substance such as sodium hydroxide, ethylenediamine, ethanolamine, sodium bicarbonate, arginine, meglumine, or trisaminomethane is added. Pharmaceutical preparations such as solutions, tablets granules or capsules can be formed with these components. Compositions for slow release of the compound can be formed as described in U.S. Pat. No. 4,880,830.

Generally, the oral administration methods and formulations of the present invention provide between about 1 mg and about 200 mg per day, for example between about 10 mg and about 100 mg per day, or between about 20 mg and about 60 mg per day, administered about 2 to about 4 times daily, of the (3R,4R)-Δ⁸—THC-11-oic acids (i.e., excluding excipients, carriers, and any of the optional additional active ingredients described herein). If desired, the daily dose may include two or more unit doses, i.e., tablets, cachets or capsules, to be administered each day.

It is further recommended that children, patients aged over 65 years, and those with impaired renal or hepatic function initially receive low doses, and that they then be titrated based on individual response(s) or blood level(s). It may be necessary to use dosages outside these ranges in some cases, as will be apparent to those of ordinary skill in the art. Further, it is noted that the clinician or treating physician will know how and when to interrupt, adjust, or terminate therapy in conjunction with individual patient response.

The methods of the present invention envision the optional inclusion of existing treatments for nausea in conjunction with the methods of administration and formulation of compounds and pharmaceutical compositions comprising the (3R,4R)-Δ⁸—THC-11-oic acids of the present invention.

Pharmaceutical compositions for use in the methods of the present invention suitable for oral administration may be presented as discrete units such as capsules, cachets, or tablets, or aerosol sprays, each containing a predetermined amount of the active ingredient, as a powder or granules, as creams, pastes, gels, or ointments, or as a solution or a suspension in an aqueous liquid, a non-aqueous liquid, an oil-in-water emulsion, or a water-in-oil liquid emulsion. Formulations that include micelles are also contemplated. Such compositions may be prepared by any of the methods of pharmacy, but all methods include the step of bringing into association the carrier with the active ingredient which constitutes one or more necessary ingredients. In general, the compositions are prepared by uniformly and intimately admixing the active ingredient with liquid carriers or finely divided solid carriers or both, and then, if necessary, shaping the product into the desired presentation.

For example, a tablet may be prepared by compression or molding, optionally, with one or more accessory ingredients. Compressed tablets may be prepared by compressing in a suitable machine the active ingredient in a free-flowing form, such as powder or granules, optionally mixed with a binder (e.g., carboxymethylcellulose, gum arabic, gelatin), filler (e.g., lactose), adjuvant, flavoring agent, coloring agent, lubricant, inert diluent, coating material (e.g., wax or plasticizer), and a surface active or dispersing agent. Molded tablets may be made by molding, in a suitable machine, a mixture of the powdered compound moistened with an inert liquid diluent. Those skilled in the art will know, or will be able to ascertain with no more than routine experimentation, appropriate pharmacological carriers for said pharmaceutical compositions.

When the compositions and pharmaceuticals according to the present invention are administered using the IV method, typically they can be provided as dispersions, suspensions, or solutions.

The methods of the present invention include the determination of optimum doses of the compounds and pharmaceutical compositions for treating nausea symptoms, which may be determined in consideration of the results of animal experiments. More specific doses obviously vary depending on the administration method, the condition of the subject such as age, body weight, sex, sensitivity, food eaten, dosage intervals, medicines administered in combination, and the seriousness and degree of the nausea. The optimal dose and the administration frequency under a given condition must be determined by the appropriate dosage test of a medical specialist based on the aforementioned guidelines, and does not constitute undue experimentation for one skilled in the art.

4. Examples

The invention is further defined by reference to the following examples describing in detail the preparation of the compounds and compositions for treating nausea according to the present invention The examples are representative and should not be construed to limit the scope of the invention.

a. Preparation of Derivatives

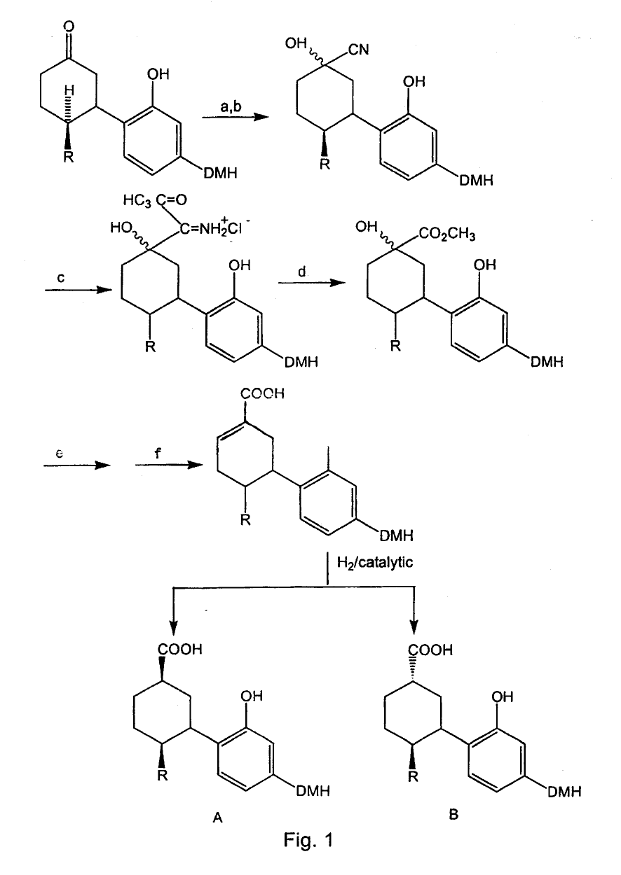

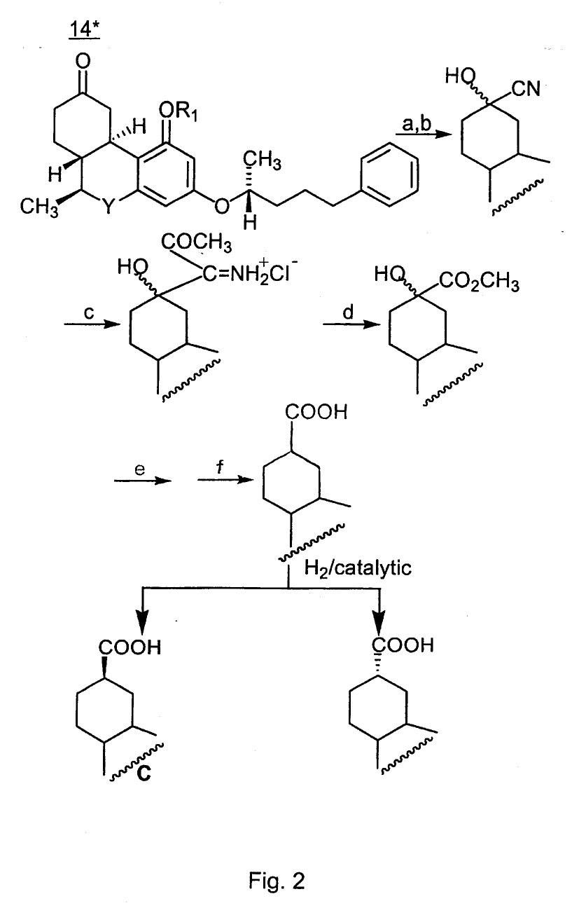

The compounds of the present invention may be prepared according to the synthetic schemes depicted in FIGS. 1 and 2.

FIG. 1 depicts a scheme to produce compounds of Formula IV, and FIG. 2 depicts a scheme to produce compounds of Formula V. DMH is dimethylheptyl in the figures, where 1′,1′-dimethylheptyl is used in the preparation of the compounds and compositions of the present invention. The intermediates and final compounds in these schemes are generally prepared by the methods disclosed in Schwartz, A., and Madan, P., J. Org. Chem., 51:5463-5465 (1986), which is expressly incorporated by reference thereto for the purpose of teaching a skilled artisan how to prepare the compounds of the present invention.

b. Various Compounds of the Invention

The following table illustrates various specific embodiments of the compounds of Formula III of the present invention. When Y is nil, R equals R³ in the table below.

TABLE 1

| | Compound | | | |
|---|---|---|---|---|
| | R¹ | R² | R³ | Y |
| 1 | hydrogen | DMH | hydrogen | nil |
| 2 | hydrogen | DMH | CH₃— | nil |
| 3 | hydrogen | DMH | CH₃CH₂— | nil |
| 4 | hydrogen | DMH | CH₃CH₂CH₂— | nil |
| 5 | hydrogen | DMH | —CH₂OH | nil |
| 6 | hydrogen | DMH | —(CH₂)₂OH | nil |
| 7 | hydrogen | DMH | —(CH₂)₃OH | nil |
| 8 | hydrogen | DMH | —(CH₂)₄OH | nil |
| 9 | hydrogen | DMH | —(CH₂)₅OH | nil |
| 10 | hydrogen | —OCHCH₃(CH₂)₃Ph | —CHCH₃ | oxygen |
| 11 | —COCH₃ | —OCHCH₃(CH₂)₃Ph | —CHCH₃ | oxygen |
| 12 | —COCH₂CH₃ | —OCHCH₃(CH₂)₃Ph | —CHCH₃ | oxygen |
| 13 | hydrogen | —OCHCH₃(CH₂)₃Ph | —CHCH₃ | NH |
| 14 | —COCH₃ | —OCHCH₃(CH₂)₃Ph | —CHCH₃ | NH |
| 15 | —COCH₂CH₃ | —OCHCH₃(CH₂)₃Ph | —CHCH₃ | NH |

DMH = 1′,1′ dimethylheptyl;
Ph = Phenyl

c. Preparation of Capsules

A large number of unit capsules are prepared by filling standard two-piece hard gelatin capsules each with the desired amount of the powdered active ingredient as described above, 150 milligrams of lactose, 50 milligrams of cellulose, and 6 milligrams magnesium stearate. The capsules may also be prepared to include existing compounds useful in treating nausea.

d. Preparation of Soft Gelatin Capsules

A mixture of active ingredient in a digestible oil such as soybean oil, lecithin, cottonseed oil or olive oil is prepared and injected by means of a positive displacement pump into gelatin to form soft gelatin capsules containing the desired amount of the active ingredient. The capsules are washed and dried for packaging. The soft gelatin capsules may also be prepared to include existing compounds useful in treating nausea.

e. Preparation of IV Formulation

A formulation suitable for intravenous administration is prepared by dissolving the desired amount of the active ingredient as described above in a suitable volume of saline. The formulation may also be prepared to include existing compounds useful in treating nausea. For instance, because the active ingredient may be relatively insoluble in water, it may be advantageously incorporated into liposomes.

5. Conclusion

Those skilled in the art will recognize, or be able to ascertain using no more than routine experimentation, many equivalents to the specific embodiments of the invention described herein. Such equivalents are intended to be encompassed by the following claims.

What is claimed is:

1. A method of treating mammals suffering from nausea with a compound of Formula III 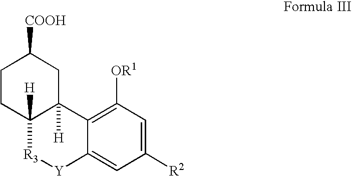
wherein R¹ is hydrogen, —COCH₃ or —COCH₂CH₃; R² is a branched C₅-C₁₂ alkyl group which may optionally have a terminal aromatic ring, or optionally a branched —OCHCH₃(CH₂)ₘ alkyl group which may have a terminal aromatic ring, wherein m is 0 to 7; and R³ is hydrogen, a C₁₋₈ alkyl or a C₁₋₈ alkanol group; and Y is nil or a bridging group of NH or oxygen; provided that where Y is oxygen and R² is a branched C₅-C₁₂ alkyl, R³ is not —CHCH₃, or a pharmaceutically acceptable salt, ester, or solvate thereof, the method comprising:
identifying a mammal suffering from or expected to suffer from nausea, and
administering to the mammal an effective amount of a compound of Formula

2. A method according to claim 1, wherein R¹ is hydrogen, and R² is 1′, 1′-dimethylheptyl.

3. A method according to claim 1, wherein R² is a branched —OCHCH₃(CH₂)ₘ alkyl group terminated with a phenyl ring, wherein m is 0 to 7, and R³ is —CHCH₃.

4. The method of claim 1, wherein the mammal is a human.

5. The method of claim 1, wherein the compound is administered orally.

6. The method of claim 1, wherein the compound is administered intravenously.

7. The method of claim 1, wherein the compound is administered via an implant.

8. The method of claim 7, wherein the implant provides slow release of the compound.

9. The method of claim 1, wherein the compound is administered in a tablet.

10. A pharmaceutical composition for use in treating nausea in a mammal, comprising:
a compound having Formula III below: 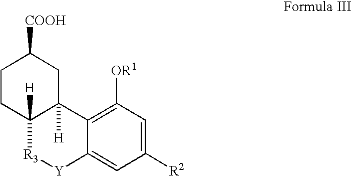
wherein R¹ is hydrogen, —COCH₃ or —COCH₂CH₃; R² is a branched C₅-C₁₂ alkyl compound which may optionally have a terminal aromatic ring, or optionally a branched —OCHCH₃(CH₂)ₘ alkyl group, which may have a terminal aromatic ring, wherein m is 0 to 7; and R³ is hydrogen, a C₁₋₈ alkyl or a C₁₋₈ alkanol group; and Y is nil or a bridging group of NH or oxygen; provided that where Y is oxygen and R² is a branched C₅-C₁₂ alkyl , R³ is not —CHCH₃; or a pharmaceutically acceptable salt, ester, or solvate thereof.

11. The pharmaceutical composition of claim 10, further comprising an anticholinergic agent selected from the group consisting of anisotropine, aprophen, artane, atropine, belladonna, benactyzine, benztropine, clidinium, dicyclomine, glycopyrrolate, homatropine, hyoscyamine, isopropamide, mepenzolate, methantheline, methscopolamine, oxybutynin, oxyphencyclimine, propantheline, scopolamine, terodiline, tridihexethyl, trihexyphenidyl, and trospium.

12. The pharmaceutical composition of claim 10, further comprising an agent useful in relieving symptoms of nausea selected from the group consisting of meclizine, sugar and phosphoric acid, hydroxizine and dimenhydrinate.

13. The pharmaceutical composition of claim 10, wherein said pharmaceutical composition is formulated for oral administration.

14. The pharmaceutical composition of claim 10, wherein said pharmaceutical composition is formulated for intravenous administration.

15. A pharmaceutical composition for use in treating nausea in a mammal, comprising:
a compound having Formula IV 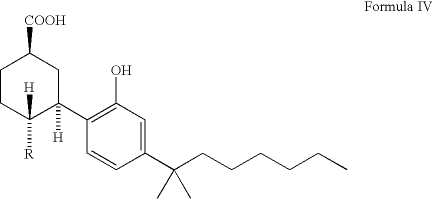
wherein R is hydrogen, branched or unbranched C₁₋₈ alkyl, and branched or unbranched C₁₋₈ alkanol; or a pharmaceutically acceptable salt, ester, or solvate thereof.

16. The pharmaceutical composition of claim 15, wherein R is selected from methyl, methanol, branched or unbranched ethyl, propyl, ethanol, and propanol.

17. A pharmaceutical composition for use in treating nausea in a mammal, comprising:
a compound having Formula V 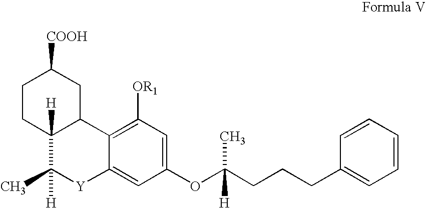
wherein R¹ is hydrogen, —COCH₃ or —COCH₂CH₃, and Y is NH or oxygen, or a pharmaceutically acceptable salt, ester, or solvate thereof.
